(19) United States
(12) Patent Application Publication
(10) Pub. No.: US 2015/0031702 A1
(43) Pub. Date: Jan. 29, 2015

(54) PI3K INHIBITORS FOR TREATING COUGH

(71) Applicant: GlaxoSmithkline Intellectual Property (No.2) Limited, Brentford, Middlesex (GB)

(72) Inventor: Emilio Merlo Pich, Brentford (GB)

(21) Appl. No.: 14/376,686

(22) PCT Filed: Feb. 4, 2013
(86) PCT No.: PCT/EP2013/052113

§ 371 Date: Aug. 5, 2014

Related U.S. Application Data
(60) Provisional application No. 61/595,299, filed on Feb. 6, 2012.

(51) Int. Cl. C07D 401/14

(52) U.S. Cl. CPC C07D 401/14
USPC 514/252.04

(57) ABSTRACT

The present invention is directed to compounds or pharmaceutically acceptable salts thereof for use in the treatment of cough, in particular idiopathic chronic cough, cough variant asthma, cough associated with thoracic tumour or lung cancer, viral and post-viral cough, upper airways cough syndrome (UACS) or post nasal drip cough, and cough associated with disorders such as gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis and infection (such as whooping cough.

### FIELD OF THE INVENTION

The present invention is directed to compounds and pharmaceutically acceptable salts thereof which are inhibitors of the activity or function of the phosphoinositide 3′OH kinase family (hereinafter PI3K), which includes PI3Kα, PI3Kβ, PI3Kγ and PI3Kδ, and the mammalian target of rapamycin (hereinafter mTOR), a PI3K downstream signalling target, for use in the treatment of cough, in particular idiopathic chronic cough, cough variant asthma, cough associated with thoracic tumour or lung cancer, viral and post-viral cough, upper airways cough syndrome (UACS) or post nasal drip cough, and cough associated with disorders such as gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis and infection (such as whooping cough).

### BACKGROUND OF THE INVENTION

Cough is an airway defensive reflex facilitating clearance of accumulated secretions and protecting airways and lungs from aspiration, inhaled particulates and irritants. However, when associated with disease, coughing can be a distressing symptom significantly affecting patient's lifestyle and wellbeing [French, 1998]. The marked decrease in health-related quality of life is responsible for cough being the most common symptom bringing patients to medical attention [Cherry, 2008] and indeed is one of the primary causes for patients with as yet undiagnosed IPF to seek medical assistance.

Cough can be subdivided into acute cough lasting for less than 3 weeks, sub-acute cough lasting between 3 and 8 weeks and chronic cough lasting for more than 8 weeks. Acute cough is most frequently associated with upper respiratory infection and although usually self-limiting, both prescription and over the counter (OTC) medication are commonly used to treat it with limited success [Smith, 2010]. Chronic cough is a common symptom of respiratory conditions such as chronic obstructive pulmonary disease (COPD), asthma, upper airways cough syndrome, idiopathic pulmonary fibrosis and some non respiratory conditions such as gastro oesophageal reflux disease. Whilst it is clear that if the underlying disease is identified and appropriately treated, the cough will often disappear, there remains a significant cohort of patients for whom no specific cause of the cough can be found, despite detailed investigations. Indeed it has been reported that chronic non-productive cough having no identifiable cause can account for up to 40% of patients presenting to special cough clinics [Irwin, 1981]. In those patients heightened cough reflex sensitivity is persistent and their condition falls into a category of unexplained (idiopathic) cough [Irvin, 2010], more recently defined as cough hypersensitivity syndrome [Morice, 2011].

The underlying mechanism leading to unexplained “hypersensitive” cough is unknown, but converging findings indicate the sensitization of “nociceptive” sensory neurons involved in respiratory reflexes as a likely substrate [Kollarik, 2010]. Cough is driven by stimuli that activates nerve terminals of vagal sensory neurons located in jugular and nodose ganglia whose terminals innervate trachea, bronchi and lungs. Sensitization of nociceptors can be induced by repeated exposure to naturally occurring stimuli or to pathologic conditions, as observed in inflammation and damage, or in chronic exposure to irritants, neurotrophic factors or neuroactive drugs [Berger, 2011]. This process, common to most neuron types, is characterized by excessive functional response to stimuli and by morphologic changes of soma, dendrites and synaptic spines, and was also recently observed in vagal sensory neurons [Lieu, 2011]. Phosphorylated PI3K is known to be involved in neuronal morphogenesis. Accordingly, phosphorylated PI3K activates the mTOR complexes and increases soma size [Kwon, 2003] and dendrite arborisation [Jaworski, 2005]. Indirect activation of PI3K phosphorylation observed with neurons of null mutant mice for PTEN was associated with mTOR activation and the increase of dendrites in the hippocampus [Kwon et al., 2006]. Agents inducing sensitization of DRG nociceptors like ephrinB activate PI3K signalling and thus PI3K inhibitors have been shown to reverse sensitization and pain in mice [Guan, 2010].

A PI3K inhibitor may be capable of inhibiting the sensitization occurring in sensory vagal neurons of subjects with chronic cough, resulting in therapeutic effects.

Since persistent cough is often debilitating and embarrassing, there is a clear need for an effective antitussive agent. The majority of available antitussive treatments have not demonstrated efficacy in placebo controlled clinical studies [Schroeder, 2002; Smith, 2006]. Whilst some of the mechanisms underlying cough are now better understood, no real progress has been made in the treatment of this condition and the need for safe and effective antitussive treatment has been highlighted by several authors [Dicpinigaitis, 2011; McGarvey, 2010; Chung, 2008].

Particular patient populations which may benefit from treatment include: 1) IPF patients with chronic cough, in which overactive PI3K pathways could be possibly present in both the fibroblasts of the active zone [Lu, 2010] and the vagal nociceptors of the lungs; 2) idiopathic chronic cough patients, characterized by a sensitized status to environmental and endogenous irritants; 3) chronic cough variant asthma patients, whose cough and bronchial hyperreactivity could be generated by an inflammatory-mediated sensitization of the vala nociceptors; and 4) lung cancer patients with cough, whose local pathologic tissue conditions are characterized by a blend of inflammatory mediators, neurotrophic factors and necrosis products that could drive a constant PI3K elevated signal in lung/broncchi nociceptors.

There remains a need to provide compounds which are inhibitors of the activity or function of PI3K and mTOR which may be useful in the treatment of cough.

### REFERENCES

French C, Irwin R S, Curley F J, et al. Impact of chronic cough on quality of life. Arch. Intern. Med. 1998; 158:1657-61.
Cherry D K, Hing E, Woodwell D A, Rechtsteiner E A. National Ambulatory Medical Care Survey: 2006 summary. Natl. Health Stat. Report 2008 (3):1-39.
Smith S M, Schroeder K, Fahey T. Over-the-counter (OTC) medications for acute cough in children and adults in ambulatory settings. The Cochrane Library 2010, Issue 9:1-33.
Irvin R S, Corrao W M, Pratter M R. Chronic persistent cough in the adult: the spectrum and frequency of causes and successful outcome of specific therapy. Am. Rev. Respir. Dis. 1981; 123: 413-417.
Irvin R S. Unexplained cough in the adult. Otolaryngol. Clin. N. Am. 2010; 43:167-180.
Morice A H, Faruqi S, Wright C E, Thompson R, Bland J M. Cough Hypersensitivity syndrome: a distinct clinical entity. Lung 2011 189(1):73-9.
Kollarik M, Ru F, Brozmanova M. Vagal afferent nerves with the properties of nociceptors. Autonomic Neuroscience: Basic and Clinical 2010 153:1-2 (12-20).
Berger J V, Knaepen L, Janssen S P M, Jaken R J P, Marcus M A E, Joosten E A J, Deumens R. Cellular and molecular insights into neuropathy-induced pain hypersensitivity for mechanism-based treatment approaches. Brain Research Reviews 2011; 67(1-2): 282-310.
Lieu T and Undem B J. Neuroplasticity in vagal afferent neurons involved in cough. Pulmonary Pharmacology and Therapeutics 2011 24:3 (276-279).
Kwon C H, Zhu X, Zang J, Baker S J. mTOR is required for hypertrophy of PTEN-deficient neuronal soma in vivo. Proc Natl Acad Sci USA 2003; 100:12923-28.
Jaworski J, Splanger S, Seeburg D P, Hoogenraad C C, Sheng M. Control of dendritic arborization by the phosphoinositide-3′-kinase-Akt-mammalian target of rapamycin pathway. J. Neurosci. 2005 25, 11300-11312.
Kwon C H et al., PTEN regulates neuronal arborisation and social interaction in mice. Neuron 2006 50:377-88.
Guan X-H, Lu X-F, Zhang H-X, Wu J-R, Yuan Y, Bao Q, Ling D-Y, Cao J-L. Phosphatidylinositol 3-kinase mediates pain behaviors induced by activation of peripheral ephrinBs/EphBs signaling in mice. Pharmacology Biochemistry and Behavior 2010 95:3 (315-324).
Schroeder K, Fahey T. Systematic review of randomised controlled trials of over-the-counter medicines for acute cough in adults. Br. Med. J. 2002; 324:329-31.
Smith J, Owen E, Earis J, Woodcock A. Effect of codeine on objective measurement of cough in chronic obstructive pulmonary disease. J. Allergy Clin. Immunol. 2006; 117(4):831-5.
Dicpinigaitis P V. Cough: an unmet clinical need. British Journal of Pharmacology 2011; 163:116-124.
McGarvey L P A, Elder J. Future directions in treating cough. Otolaryngol. Clin. N. Am. 2010; 43:199-211.
Chung K F, Pavord I D. Prevalence, pathogenesis and causes of chronic cough. Lancet. 2008; 371:1364-74.
Lu Y, Azad N, Wang L, Iyer A K V, Castranova V, Jiang B H, Rojanasakul, Y. Phosphatidylinositol-3-kinase/akt regulates bleomycin-induced fibroblast proliferation and collagen production. Am. J. Respir. Cell Mol. Biol. 2010; 42:432-441.

### SUMMARY OF THE INVENTION

The present invention provides a compound of formula (I)

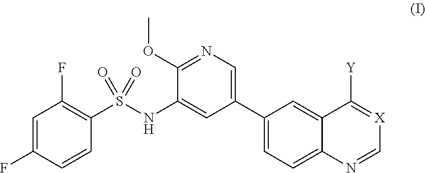

wherein
X is —CH— and Y is 4-pyridazinyl; or
X is —N— and Y is 4-morpholinyl;
or a pharmaceutically acceptable salt thereof for use in the treatment of cough.

### BRIEF DESCRIPTION OF THE FIGURES

FIG. 1 shows the concentration dependence of 2,4-difluoro-N-{2-(methyloxy)-5-[4-(4-morpholinyl)-6-quinazolinyl]-3-pyridinyl}benzenesulfonamide (GSK-1) in a sensitization assay.

### DETAILED DESCRIPTION OF THE INVENTION

In one embodiment, the present invention provides a compound of formula (I)

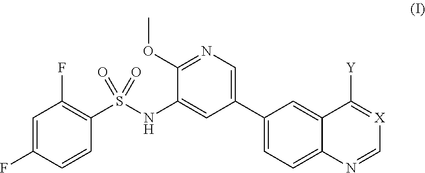

wherein
X is —CH— and Y is 4-pyridazinyl; or
X is —N— and Y is 4-morpholinyl;
or a pharmaceutically acceptable salt thereof for use in the treatment of cough.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

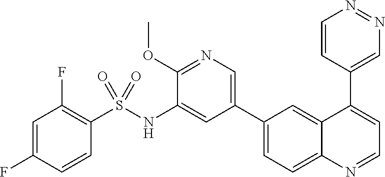 [SMILES: COc1ncc(-c2ccc3nccc(-c4ccnnc4)c3c2)cc1NS(=O)(=O)c1ccc(F)cc1F]

or a pharmaceutically acceptable salt thereof for use in the treatment of cough.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

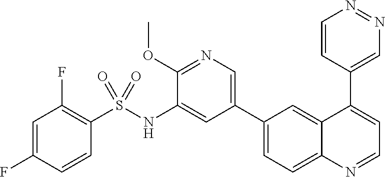 [SMILES: COc1ncc(-c2ccc3nccc(-c4ccnnc4)c3c2)cc1NS(=O)(=O)c1ccc(F)cc1F]

for use in the treatment of cough.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

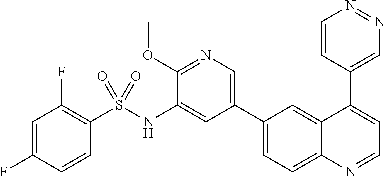 [SMILES: COc1ncc(-c2ccc3nccc(-c4ccnnc4)c3c2)cc1NS(=O)(=O)c1ccc(F)cc1F]

or a pharmaceutically acceptable salt thereof for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further embodiment, the present invention provides a compound which is 2,4-difluoro-N-{2-(methyloxy)-5-[4-(4-pyridazinyl)-6-quinolinyl]-3-pyridinyl}benzenesulfonamide:

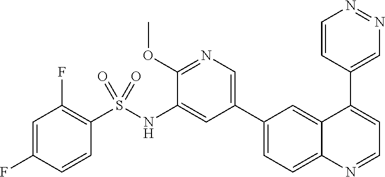 [SMILES: COc1ncc(-c2ccc3nccc(-c4ccnnc4)c3c2)cc1NS(=O)(=O)c1ccc(F)cc1F]

for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

Included within the scope of the invention is the use of all solvates (including hydrates), complexes, polymorphs, prodrugs and radiolabelled derivatives of a compound of formula (I) or a pharmaceutically acceptable salt thereof.

Compounds of formula (I) may be administered as a pharmaceutically acceptable salt. As used herein, the term “pharmaceutically acceptable salt” refers to a salt that retains the desired biological activity of the compound and exhibits minimal undesired toxicological effects. Pharmaceutically acceptable salts of compounds may be used to impart greater stability or solubility to a molecule thereby facilitating formulation into a dosage form. These pharmaceutically acceptable salts may be prepared in situ during the final isolation and purification of the compound, or by separately reacting the purified compound, or a non-pharmaceutically acceptable salt thereof, with a suitable base or acid. For a review on suitable salts see Berge et al., J. Pharm. Sci., 1977, 66, 1-19. In one embodiment, the invention provides the use of a pharmaceutically acceptable salt of 2,4-difluoro-N-{2-(methyloxy)-5-[4-(4-pyridazinyl)-6-quinolinyl]-3-pyridinyl}benzenesulfonamide. In a further embodiment, the invention provides the use of 2,4-difluoro-N-{2-(methyloxy)-5-[4-(4-pyridazinyl)-6-quinolinyl]-3-pyridinyl}benzenesulfonamide as the free base.

### Compound Preparation

The compounds for use according to the invention may be made by a variety of methods, including standard chemistry. For example, 2,4-difluoro-N-{2-(methyloxy)-5-[4-(4-pyridazinyl)-6-quinolinyl]-3-pyridinyl}benzenesulfonamide may be prepared as described in WO 2008/144463 and 2,4-difluoro-N-{2-(methyloxy)-5-[4-(4-morpholinyl)-6-quinazolinyl]-3-pyridinyl}benzenesulfonamide may be prepared as described in WO 2008/157191.

### Methods of Use

The methods of treatment of the invention comprise administering a safe and effective amount of a compound of formula (I) or a pharmaceutically acceptable salt thereof to a patient in need thereof.

As used herein, “treat” in reference to a disorder means: (1) to ameliorate the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As used herein, “safe and effective amount” in reference to a compound of formula (I) or a pharmaceutically acceptable salt thereof, or other pharmaceutically-active agent, means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

The compound or a pharmaceutically acceptable salt thereof may be administered by any suitable route of administration, in particular oral administration.

The compound or a pharmaceutically acceptable salt thereof may be administered according to a dosing regimen wherein a number of doses are administered at varying intervals of time for a given period of time. For example, doses may be administered one, two, three, or four times per day. In one embodiment, a dose is administered twice per day (BID).

Doses may be administered until the desired therapeutic effect is achieved or indefinitely to maintain the desired therapeutic effect. Suitable dosing regimens, including the duration such regimens are administered, may depend on the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages for oral administration may range from about 0.1 mg to about 20 mg, for example from about 0.1 mg to about 10 mg such as about 0.4 mg to about 7 mg. For example, a dose of from about 0.1 mg to about 5 mg, for example from about 0.2 mg to about 3.5 mg such as from about 0.25 mg to about 3 mg, may be administered BID per patient. In one embodiment, a dose of from about 0.25 mg to about 2.5 mg may be administered BID per patient.

In one aspect, the invention provides a compound of formula (I) or a pharmaceutically acceptable salt thereof for use in the treatment of cough.

Cough treated according to the invention may be chronic cough associated with sensitization. In particular, cough may be idiopathic chronic cough, cough variant asthma, cough associated with thoracic tumour or lung cancer, viral or post-viral cough, upper airways cough syndrome (UACS) or post nasal drip cough, or cough associated with disorders such as gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough). In one embodiment, cough may be idiopathic chronic cough, cough variant asthma, cough associated with thoracic tumour or lung cancer, viral or post-viral cough, or cough associated with disorders such as chronic obstructive pulmonary disease (COPD), interstitial lung disease (such as idiopathic pulmonary fibrosis (IPF)), congestive heart disease, sarcoidosis or infection (such as whooping cough). In another embodiment, cough may be idiopathic chronic cough, cough variant asthma, cough associated with thoracic tumour or lung cancer, or cough associated with disorders such as interstitial lung disease (such as idiopathic pulmonary fibrosis (IPF). In another embodiment, cough may be idiopathic chronic cough, cough associated with thoracic tumour or lung cancer, or cough associated with disorders such as chronic obstructive pulmonary disease (COPD), interstitial lung disease (such as idiopathic pulmonary fibrosis (IPF)) or sarcoidosis. In another embodiment, cough may becough associated with thoracic tumour or lung cancer, or cough associated with interstitial lung disease (such as idiopathic pulmonary fibrosis (IPF)). In a further embodiment, cough may be cough associated with idiopathic pulmonary fibrosis (IPF).

In one embodiment, the invention provides the use of a compound of formula (I) or a pharmaceutically acceptable salt thereof in the manufacture of a medicament for use in the treatment of cough.

In a further embodiment, the invention provides a method of treating cough comprising administering a safe and effective amount of a compound of formula (I) or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof for use in the treatment of cough.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof in the manufacture of a medicament for use in the treatment of cough.

In a further embodiment, the invention provides a method of treating cough comprising administering a safe and effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide for use in the treatment of cough.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide in the manufacture of a medicament for use in the treatment of cough.

In a further embodiment, the invention provides a method of treating cough comprising administering a safe and effective amount of 2,4-difluoro-N-{2-(methyloxy)-5-[4-(4-pyridazinyl)-6-quinolinyl]-3-pyridinyl}benzenesulfonamide to a patient in need thereof.

In another aspect, the invention provides a compound of formula (I) or a pharmaceutically acceptable salt thereof for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In one embodiment, the invention provides the use of a compound of formula (I) or a pharmaceutically acceptable salt thereof in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of a compound of formula (I) or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In one embodiment, the invention provides the use of a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof to a patient in need thereof.

In a further aspect, the invention provides a compound which is 2,4-difluoro-N-{2-(methyloxy)-5-[4-(4-pyridazinyl)-6-quinolinyl]-3-pyridinyl}benzenesulfonamide for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In one embodiment, the invention provides the use of a compound which is 2,4-difluoro-N-{2-(methyloxy)-5-[4-(4-pyridazinyl)-6-quinolinyl]-3-pyridinyl}benzenesulfonamide in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of 2,4-difluoro-N-{2-(methyloxy)-5-[4-(4-pyridazinyl)-6-quinolinyl]-3-pyridinyl}benzenesulfonamide to a patient in need thereof.

### Compositions

Compounds of formula (I) or pharmaceutically acceptable salts thereof may be formulated into a pharmaceutical composition prior to administration to a patient.

Accordingly, in one aspect the invention is directed to pharmaceutical compositions comprising a compound of formula (I) or a pharmaceutically acceptable salt thereof, and one or more pharmaceutically acceptable excipients for use in the treatment of cough.

In one embodiment, the invention is directed to pharmaceutical compositions comprising a compound of formula (I) or a pharmaceutically acceptable salt thereof, and one or more pharmaceutically acceptable excipients for use in the treatment of idiopathic chronic cough, cough variant asthma, cough associated with thoracic tumour or lung cancerviral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide or a pharmaceutically acceptable salt thereof, and one or more pharmaceutically acceptable excipients for use in the treatment of cough.

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide or a pharmaceutically acceptable salt thereof, and one or more pharmaceutically acceptable excipients for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide, and one or more pharmaceutically acceptable excipients for use in the treatment of cough.

In a further embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide, and one or more pharmaceutically acceptable excipients for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another aspect the invention is directed to pharmaceutical compositions comprising 0.1 mg to about 5 mg of a compound of formula (I) or a pharmaceutically acceptable salt thereof and about 0.1 g to about 2 g of one or more pharmaceutically acceptable excipients for use in the treatment of cough.

In one embodiment, the invention is directed to pharmaceutical compositions comprising 0.1 mg to about 5 mg of a compound of formula (I) or a pharmaceutically acceptable salt thereof and about 0.1 g to about 2 g of one or more pharmaceutically acceptable excipients for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide or a pharmaceutically acceptable salt thereof and about 0.1 g to about 2 g of one or more pharmaceutically acceptable excipients for use in the treatment of cough.

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide or a pharmaceutically acceptable salt thereof and about 0.1 g to about 2 g of one or more pharmaceutically acceptable excipients for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide and about 0.1 g to about 2 g of one or more pharmaceutically acceptable excipients for use in the treatment of cough.

In a further embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide and about 0.1 g to about 2 g of one or more pharmaceutically acceptable excipients for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further aspect the invention is directed to a pharmaceutical composition for the treatment of cough comprising a compound of formula (I) or a pharmaceutically acceptable salt thereof.

In one embodiment, the invention is directed to a pharmaceutical composition for the treatment of cough comprising 2,4-difluoro-N-{2-(methyloxy)-5-[4-(4-pyridazinyl)-6-quinolinyl]-3-pyridinyl}benzenesulfonamide or a pharmaceutically acceptable salt thereof.

In another embodiment, the invention is directed to a pharmaceutical composition for the treatment of cough comprising 2,4-difluoro-N-{2-(methyloxy)-5-[4-(4-pyridazinyl)-6-quinolinyl]-3-pyridinyl}benzenesulfonamide.

In another embodiment, the invention is directed to a pharmaceutical composition for the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising a compound of formula (I) or a pharmaceutically acceptable salt thereof.

In another embodiment, the invention is directed to a pharmaceutical composition for the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising 2,4-difluoro-N-{2-(methyloxy)-5-[4-(4-pyridazinyl)-6-quinolinyl]-3-pyridinyl}benzenesulfonamide or a pharmaceutically acceptable salt thereof.

In further embodiment, the invention is directed to a pharmaceutical composition for the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising 2,4-difluoro-N-{2-(methyloxy)-5-[4-(4-pyridazinyl)-6-quinolinyl]-3-pyridinyl}benzenesulfonamide.

The pharmaceutical compositions for use according to the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions for use according to the invention may be prepared and packaged in unit dosage form wherein each physically discrete unit contains a compound of formula (I) or a pharmaceutically acceptable salt thereof. When prepared in unit dosage form, the pharmaceutical compositions for use according to the invention typically may contain, for example, from about 0.1 mg to about 5 mg, for example from about 0.2 mg to about 3.5 mg such as from about 0.25 mg to about 3 mg of a compound of formula (I) or a pharmaceutically acceptable salt thereof. In one embodiment, the pharmaceutical compositions for use according to the invention typically contain about 0.25 mg of a compound of formula (I) or a pharmaceutically acceptable salt thereof. In a further embodiment, the pharmaceutical compositions for use according to the invention typically contain about 0.5 mg of a compound of formula (I) or a pharmaceutically acceptable salt thereof.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of a compound of formula (I) or a pharmaceutically acceptable salt thereof when administered to a patient and interactions which would result in pharmaceutical compositions that are not pharmaceutically acceptable are avoided. In addition, each excipient must of course be pharmaceutically-acceptable eg of sufficiently high purity.

The compounds of formula (I) or pharmaceutically acceptable salts thereof and the pharmaceutically acceptable excipient or excipients will typically be formulated into a dosage form adapted for administration to the patient by the desired route of administration. For example, dosage forms include those adapted for oral administration such as tablets, capsules, caplets, pills, troches, powders, syrups, elixers, suspensions, solutions, emulsions, sachets, and cachets), or those adapted for inhalation such as aerosols, solutions, and dry powders.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of a compound of formula (I) or a pharmaceutically acceptable salt thereof once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions for use according to the invention are prepared using techniques and methods known to those skilled in the art. Some of the methods commonly used in the art are described in Remington's Pharmaceutical Sciences (Mack Publishing Company).

A pharmaceutical composition comprising a compound of formula (I) or a pharmaceutically acceptable salt thereof may be prepared by, for example, admixture at ambient temperature and atmospheric pressure.

In one embodiment, a compound of formula (I) or a pharmaceutically acceptable salt thereof will be formulated for oral administration. In a further embodiment, the compounds of formula (I) or pharmaceutically acceptable salts thereof will be formulated for inhaled administration.

In one aspect, the composition for use according to the invention is a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compound of formula (I) or a pharmaceutically acceptable salt thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compound of formula (I) or a pharmaceutically acceptable salt thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the composition for use according to the invention is a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving a compound of formula (I) or a pharmaceutically acceptable salt thereof in a suitably flavored aqueous solution, while elixirs are prepared through the use of a non-toxic alcoholic vehicle. Suspensions can be formulated by dispersing a compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation, for example as a dry powder, an aerosol, a suspension, or a solution composition. In one embodiment, the invention is directed to a dosage form adapted for administration to a patient by inhalation as a dry powder. In a further embodiment, the invention is directed to a dosage form adapted for administration to a patient by inhalation via a nebulizer.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or pharmaceutically acceptable salt thereof. Alternatively, the compound of formula (I) or pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

According to the invention, a compound of formula (I) or a pharmaceutically acceptable salt thereof may be used in combination with one or more other therapeutic agents, in the treatment of cough.

Suitable therapeutic agents for use in combination with a compound of formula (I) or a pharmaceutically acceptable salt thereof include adjunctive cough medications such as morphine, SR morphine, codeine, hydrocodone and dextromethorphan.

The invention thus provides, in one aspect, a combination comprising a compound of formula (I) or a pharmaceutically acceptable salt thereof and one or more other therapeutically active agents for use in the treatment of cough.

In one embodiment, the invention provides a method of treating cough comprising administering a safe and effective amount of a combination comprising a compound of formula (I) or a pharmaceutically acceptable salt thereof and one or more therapeutically active agents.

In a further embodiment, the invention provides a combination comprising a compound of formula (I) or a pharmaceutically acceptable salt thereof and one or more therapeutically active agents in the manufacture of a medicament for use in the treatment of cough.

In another aspect, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more other therapeutically active agents for use in the treatment of cough.

In another embodiment, the invention provides a method of treating cough comprising administering a safe and effective amount of a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents in the manufacture of a medicament for use in the treatment of cough.

In another aspect, the invention provides a combination comprising 2,4-difluoro-N-{2-(methyloxy)-5-[4-(4-pyridazinyl)-6-quinolinyl]-3-pyridinyl}benzenesulfonamide and one or more other therapeutically active agents for use in the treatment of cough.

In one embodiment, the invention provides a method of treating cough comprising administering a safe and effective amount of a combination comprising 2,4-difluoro-N-{2-(methyloxy)-5-[4-(4-pyridazinyl)-6-quinolinyl]-3-pyridinyl}benzenesulfonamide and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide and one or more therapeutically active agents in the manufacture of a medicament for use in the treatment of cough.

In another aspect, the invention provides a combination comprising a compound of formula (I) or a pharmaceutically acceptable salt thereof and one or more other therapeutically active agents for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of a combination comprising a compound of formula (I) or a pharmaceutically acceptable salt thereof and one or more therapeutically active agents.

In a further embodiment, the invention provides a combination comprising a compound of formula (I) or a pharmaceutically acceptable salt thereof and one or more therapeutically active agents in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another aspect, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more other therapeutically active agents for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further aspect, the invention provides a combination comprising 2,4-difluoro-N-{2-(methyloxy)-5-[4-(4-pyridazinyl)-6-quinolinyl]-3-pyridinyl}benzenesulfonamide and one or more other therapeutically active agents for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention provides a method of treating idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough) comprising administering a safe and effective amount of a combination comprising 2,4-difluoro-N-{2-(methyloxy)-5-[4-(4-pyridazinyl)-6-quinolinyl]-3-pyridinyl}benzenesulfonamide and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide and one or more therapeutically active agents in the manufacture of a medicament for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

One embodiment of the invention provides the use of combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof and another therapeutically active agent for use in the treatment of cough.

In one embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide or a pharmaceutically acceptable salt thereof and another therapeutically active agent for use in the treatment of cough.

In another embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide and another therapeutically active agent for use in the treatment of cough.

In another embodiment, the invention provides a pharmaceutical composition comprising a combination of a compound of formula (I) or a pharmaceutically acceptable salt thereof and another therapeutically active agent for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In another embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide or a pharmaceutically acceptable salt thereof and another therapeutically active agent for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

In a further embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide and another therapeutically active agent for use in the treatment of idiopathic chronic cough, cough variant asthma, cough associated with thoracic tumour or lung cancer, viral or post-viral cough, upper airways cough syndrome (UACS) or post nasal drip cough, or cough associated gastro oesophageal reflux disease (both acid and non acid reflux), chronic bronchitis, chronic obstructive pulmonary disease (COPD), interstitial lung disease (such as idiopathic pulmonary fibrosis (IPF)), congestive heart disease, sarcoidosis or infection (such as whooping cough).

### Biological Data

Capsaicin, the active ingredient in pepper sprays, is an irritant that causes noxious respiratory sensations and reflexes including sneezing and coughing in humans. Capsaicin binds to Transient Receptor Potential Vanilloid 1 (TRPV1), whose expression is enriched in both somatic and visceral nociceptors. Exaggerated response to inhaled capsaicin can be seen in subjects with IPF or with cough hypersensitivity syndrome and can be used as a test. Increased reactivity to capsaicin is generally considered a marker of sensitization of nociceptors. Capsaicin effects in sensory neurons dissected from mouse peripheral ganglia can be used as a model to identify nociceptors and to study the sensitization mechanisms in vitro. Response to capsaicin can be quantified in a relatively large population of dissociated sensory neurons using the method of Calcium Imaging.

In a typical experiment, neurons were loaded with 5 μM Fura-2-AM-Ester containing 0.02% Pluronic F-127 and incubated for 30 to 45 min at 37° C. For baseline measurements, extracellular solution (145 mM NaCl, 1.25 mM CaCl₂, 1 mM MgCl₂, 5 mM KC, 10 mM D-glucose, 10 mM HEPES, pH 7.3) was added by bath application and revealed using a fluorescence miscroscope. Exposure to capsaicin produced activation of a subset of neurons, indicating the nociceptor phenotype.

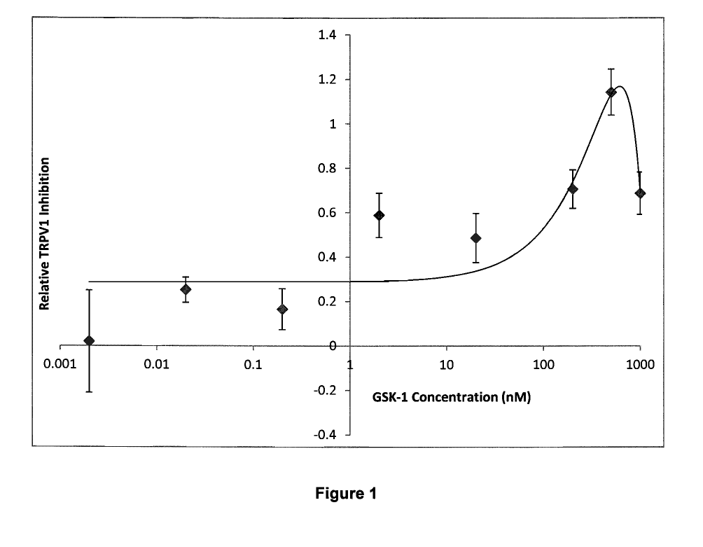

This profile of activation could be enhanced by pre-incubation with a “sensitizing cocktail” that simulates the extracellular fluid in tissue under inflammatory/damage conditions. It consisted of a mixture of cytokine, trophic factors and sensitizing factors (i.e., 10 ng/ml NGF, 2 ng/ml GDNF, 3 μM PGE2, 10 ng/ml 1β). Prolonged exposures to the “sensitizing cocktail” (from few minutes up to several hours) significantly enhanced the Calcium Imaging response to capsaicin. This effect was dose-dependently abolished by co-incubation with various doses of the compound GSK-1 (see enclosed FIG. 1) in a series of experiments. GSK-1 was 2,4-difluoro-N-{2-(methyloxy)-5-[4-(4-morpholinyl)-6-quinazolinyl]-3-pyridinyl}benzenesulfonamide.

These findings demonstrated that sensory neurons expressing TRPV1 responded to the irritant capsaicin and that the TRPV1 response can be dynamically modulated by a number of inflammatory mediators, resulting in increased sensory neuron sensitivity to capsaicin. The inhibition produced by the GSK-1 phosphatidyl inositol 3-kinase (PI3K) inhibitor indicated a critical role for the PI3K pathway in mediating sensitizing effects in sensory neurons. Published evidence indicates that PI3K is expressed in sensory neurons of the dorsal root ganglia [Zhu, 2011] and that this pathway could mediate the sensitizing effects of ephrinB as shown using commercial PI3K inhibitors in vitro, and in vivo measuring pain [Guan, 2010].

The PI3K molecular pathway is thought to underlie the elevated cough reflex sensitivity to capsaicin seen in patients with respiratory disorders, including idiopathic pulmonary fibrosis (IPF). Therefore its blockade would result in reduced sensitization of sensory neurons and reduced occurrence of cough.

Concentration Dependence of 2,4-difluoro-N-{2-(methyloxy)-5-[4-(4-morpholinyl)-6-quinazolinyl]-3-pyridinyl}benzenesulfonamide (GSK-1) on the Sensitization Assay

The graph in FIG. 1 is representative of the 3^{rd} capsaicin-induced peak in dissociated dorsal ganglion sensory neurons using Calcium Imaging (out of four consecutive peaks obtained every 10-15 min).

The curve of best fit is described by the following formula:

y=−8E−10x³−1E−06x²+0.0026x+0.2855

Each point represents how well the capsaicin-induced peak is reduced by the drug when compared with negative control (1, no sensitizing solution, no GSK-1) and positive control (0, sensitizing solution, no GSK-1). Calculated, it is:

$$\frac{C - x}{C - N}$$

Where C=the average relative f340/f380 value of peak 3 for the sensitization control (sensitizing solution, no GSK-1). X=the individual relative f340/f380 value to be calculated, and N=the average relative f340/f380 value of peak 3 for the negative control (no sensitization solution, no GSK-1).

Therefore, a score of 1 is comparable to bringing the capsaicin response back to normal calcium fluxes, a score of 0 does not reduce the sensitization induced by our inflammatory compounds, and a negative score means it actually is increasing the sensitization.

The n for each concentration is as follows:

| | |
|---|---|
| Concentration | N |

| | | |
|---|---|---|
| 0.002 | nM | 18 |
| 0.02 | nM | 48 |
| 0.2 | nM | 28 |
| 2 | nM | 7 |
| 20 | nM | 15 |
| 200 | nM | 19 |
| 500 | nM | 9 |
| 1000 | nM | 6 |

### REFERENCES

Zhu W, Oxford G S. Differential gene expression of neonatal and adult DRG neurons correlates with the differential sensitization of TRPV1 responses to nerve growth factor. Neuroscience Letters 2011 500:3 (192-196).
Guan X-H, Lu X-F, Zhang H-X, Wu J-R, Yuan Y, Bao Q, Ling D-Y, Cao J-L. Phosphatidylinositol 3-kinase mediates pain behaviors induced by activation of peripheral ephrinBs/EphBs signaling in mice. Pharmacology Biochemistry and Behavior 2010 95:3 (315-324).

1. A method of treating cough comprising administering a safe and effective amount of a compound of formula (I): 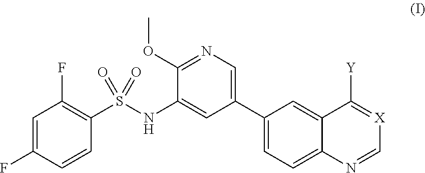
wherein
X is —CH— and Y is 4-pyridazinyl; or
X is —N— and Y is 4-morpholinyl;
or a pharmaceutically acceptable salt thereof to a patient in need thereof.

2-5. (canceled)

6. A method of treating cough comprising administering a safe and effective amount of a compound which is 2,4-difluoro-N-{2-(methyloxy)-5-[4-(4-pyridazinyl)-6-quinolinyl]-3-pyridinyl}benzenesulfonamide, represented by the structure: 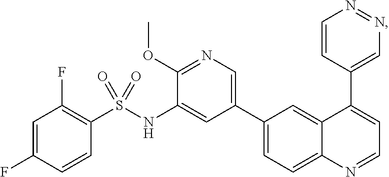 [SMILES: COc1ncc(-c2ccc3nccc(-c4ccnnc4)c3c2)cc1NS(=O)(=O)c1ccc(F)cc1F]
or a pharmaceutically acceptable salt thereof to a patient in need thereof.

7. A method of treating cough comprising administering a safe and effective amount of a compound which is 2,4-difluoro-N-{2-(methyloxy)-5-[4-(4-pyridazinyl)-6-quinolinyl]-3-pyridinyl}benzenesulfonamide, represented by the structure: 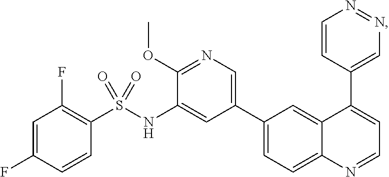 [SMILES: COc1ncc(-c2ccc3nccc(-c4ccnnc4)c3c2)cc1NS(=O)(=O)c1ccc(F)cc1F]
to a patient in need thereof.

8. The method according to claim 1 wherein the cough is idiopathic chronic cough, cough variant asthma, cough associated with thoracic tumor or lung cancer, viral or post-viral cough, upper airways cough syndrome, post nasal drip cough, or cough associated with gastro oesophageal reflux disease, chronic bronchitis, chronic obstructive pulmonary disease, interstitial lung disease, congestive heart disease, sarcoidosis or infection.

9. The method according to claim 6 wherein the cough is idiopathic chronic cough, cough variant asthma, cough associated with thoracic tumor or lung cancer, viral or post-viral cough, upper airways cough syndrome, post nasal drip cough, or cough associated with gastro oesophageal reflux disease, chronic bronchitis, chronic obstructive pulmonary disease, interstitial lung disease, congestive heart disease, sarcoidosis or infection.

10. The method according to claim 7 wherein the cough is idiopathic chronic cough, cough variant asthma, cough associated with thoracic tumor or lung cancer, viral or post-viral cough, upper airways cough syndrome, post nasal drip cough, or cough associated with gastro oesophageal reflux disease, chronic bronchitis, chronic obstructive pulmonary disease, interstitial lung disease, congestive heart disease, sarcoidosis or infection.
